(19) United States
(12) Patent Application Publication
(10) Pub. No.: US 2024/0249613 A1
(43) Pub. Date: Jul. 25, 2024

(54) Communications With External Services

(71) Applicant: APPLE INC., Cupertino, CA (US)

(72) Inventors: Manuel Roman Cuesta, Sunnyvale, CA (US); Xiaoyue Zhang, Saratoga, CA (US)

(21) Appl. No.: 18/539,339

(22) Filed: Dec. 14, 2023

Related U.S. Application Data
(60) Provisional application No. 63/481,287, filed on Jan. 24, 2023.

(51) Int. Cl. G08B 25/10; A61B 5/00; A61B 5/11

(52) U.S. Cl. CPC G08B 25/10; A61B 5/0004; A61B 5/1112; A61B 5/4064; A61B 5/6801; A61B 5/747; A61B 2562/0219

(57) ABSTRACT

A method includes determining a position of a wearable electronic device relative to the transportation device, detecting an event in which the transportation device has contacted an external object, and in response, obtaining user state information from the wearable electronic device.

### CROSS-REFERENCE TO RELATED APPLICATIONS

This application claims the benefit of, and priority to, U.S. Provisional Application No. 63/481,287, filed on Jan. 24, 2023, the content of which is hereby incorporated by reference in its entirety herein for all purposes.

### FIELD

The present disclosure relates generally to the field of communications with external services.

### BACKGROUND

Some vehicle systems are able to communicate with external services. As an example, a vehicle system may be able to communicate with an external service when the vehicle or occupants of the vehicle need assistance.

### SUMMARY

A first aspect of the disclosure is a method that includes detecting, by a control system of a transportation device, an event in which the transportation device has contacted an external object, and in response to the event, obtaining state information about a wearer of a wearable electronic device. Obtaining the state information includes obtaining motion information describing motion of the wearer of the wearable electronic device during the event, and obtaining physiological sensor information from one or more physiological sensors of the wearable electronic device, the physiological sensor information describing one or more physiological states of the wearer of the wearable electronic device. The method also includes transmitting, by the control system of the transportation device, the user state information about the wearer of the wearable electronic device to an emergency service provider system, the user state information including the motion information, and the physiological sensor information.

Some implementations of the method according to the first aspect of the disclosure further include determining, by a control system of a transportation device, a position of a wearable electronic device relative to the transportation device; and transmitting, by the control system of the transportation device, the position of the wearable electronic device relative to the transportation device to the emergency service provider system. In some implementations, determining the position of the wearable electronic device relative to the transportation device includes receiving, by the control system of the transportation device, a location signal from the wearable electronic device. Some implementations of the method according to the first aspect of the disclosure further include determining that the wearable electronic device is present in the transportation device based on a wireless communication received by the transportation device from the wearable electronic device.

In some implementations of the method according to the first aspect of the disclosure, the motion information describing motion of the user of the wearable electronic device during the event includes an acceleration value measured by an acceleration sensor of the wearable electronic device during the event. In some implementations of the method according to the first aspect of the disclosure, the physiological sensor information includes heart rate information. In some implementations of the method according to the first aspect of the disclosure, the physiological sensor information includes oxygen level information for the user of the wearable electronic device.

In some implementations of the method according to the first aspect of the disclosure, obtaining the state information further includes obtaining cognitive function information by prompting the wearer of the wearable electronic device to complete one or more tasks using the wearable electronic device. In some implementations of the method according to the first aspect of the disclosure, transmitting the user state information to the emergency service provider system comprises transmitting the cognitive function information to the emergency provider system. In some implementations of the method according to the first aspect of the disclosure, transmitting the user state information to the emergency service provider system is performed in response to determining non-compliance of the cognitive function information relative to a standard.

A second aspect of the disclosure is a non-transitory computer-readable storage device including program instructions executable by one or more processors that, when executed, cause the one or more processors to perform operations. The operations include detecting, by a control system of a transportation device, an event in which the transportation device has contacted an external object, and in response to the event, obtaining state information about a wearer of a wearable electronic device. Obtaining the state information includes obtaining motion information describing motion of the wearer of the wearable electronic device during the event, and obtaining physiological sensor information from one or more physiological sensors of the wearable electronic device, the physiological sensor information describing one or more physiological states of the wearer of the wearable electronic device. The operations also include transmitting, by the control system of the transportation device, the user state information about the wearer of the wearable electronic device to an emergency service provider system, the user state information including the motion information, and the physiological sensor information.

A third aspect of the disclosure is an apparatus that includes a memory, and one or more processors that are configured to execute instructions that are stored in the memory. The instructions, when executed, cause the one or more processors to detect, by a control system of a transportation device, an event in which the transportation device has contacted an external object, and in response to the event, obtain state information about a wearer of a wearable electronic device. The instructions to obtain the state information include instructions that cause the one or more processors to includes obtain motion information describing motion of the wearer of the wearable electronic device during the event, and to obtain physiological sensor information from one or more physiological sensors of the wearable electronic device, the physiological sensor information describing one or more physiological states of the wearer of the wearable electronic device. The instructions further cause the one or more processors to transmit, by the control system of the transportation device, the user state information about the wearer of the wearable electronic device to an emergency service provider system, the user state information including the motion information, and the physiological sensor information.

### BRIEF DESCRIPTION OF THE DRAWINGS

FIG. 1 is a block diagram of a transportation device.

FIG. 2 is a block diagram of a communications system.

FIG. 3 is a block diagram of a process for communicating with external services.

FIG. 4 is a block diagram of an example of a computing device.

### DETAILED DESCRIPTION

The disclosure herein relates to communications between a transportation device and/or wearable electronic devices that are worn by occupants of the transportation device and an external system. The external system may be, for example, an emergency service provider system. The systems and methods described herein may be used to allow information, including physiological information about occupants, to be provided to an emergency service provider system in response to an event in which the transportation device has contacted an external object.

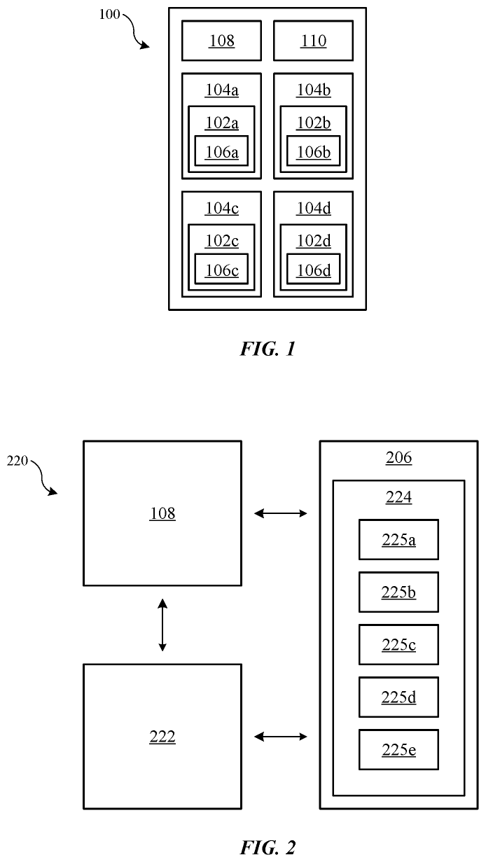

FIG. 1 is an illustration of a transportation device 100. Multiple occupants may be transported by the transportation device 100, for example, in a passenger cabin of the transportation device 100. In the illustrated example, a first occupant 102a, a second occupant 102b, a third occupant 102c, and a fourth occupant 102d are shown, but the number of occupants may vary. Each of the occupants may be at a different location within the transportation device 100. In the illustrated implementation, the first occupant 102a is sitting in a first seat 104a, the second occupant 102b is sitting in a second seat 104b, the third occupant 102c is sitting in a third seat 104c, and the fourth occupant 102d is sitting in a fourth seat 104d. Other configurations are possible.

The occupants of the transportation device 100 may each be wearing an electronic device, such as a smart watch, a fitness monitor, or another type of device. In the illustrated implementation, the first occupant 102a has a first wearable electronic device 106a, the second occupant 102b has a second wearable electronic device 106b, the third occupant 102c has a third wearable electronic device 106c, and the fourth occupant 106c has a fourth wearable electronic device 106d. For convenience, persons located in the transportation device 100, such as the first occupant 102a, the second occupant 102b, the third occupant 102c, and the fourth occupant 102d, may simply be referred to as occupants, and devices worn by them, such as the first wearable electronic device 106a, the second wearable electronic device 106b, the third wearable electronic device 106c, and the fourth wearable electronic device 106d, may simply be referred to as wearable electronic devices.

The transportation device 100 may be, as an example, a vehicle that is configured to travel on roads or other surfaces using wheels, tires, a propulsion system, a steering system, so forth. The transportation device 100 may include a control system 108 that is implemented using one or more computing devices. The transportation device 100 may also include sensors 110 that are configured to output sensor information representing conditions in the environment outside of the transportation device 100, conditions inside the passenger cabin of the transportation device 100, operating conditions of the transportation device 100, and/or other conditions. As examples, the sensors 110 may include cameras, lidar sensors, radar sensors, ultrasonic sensors, and/or other sensors that are configured to output data representing conditions of the environment outside the transportation device 100. As examples, the sensors 110 may include cameras, lidar sensors, radar sensors, ultrasonic sensors, and/or other sensors that are configured to output data representing conditions in the passenger cabin of the transportation device 100. The sensor information that is output by the sensors 110 may be provided to the control system 108 and process to interpret it, such as by identifying the presence and locations of objects, persons, and/or other features either outside or inside the transportation device 100.

The control system 108 may also be in communication with occupant devices that are present in the transportation device, such as the first wearable electronic device 106a, the second wearable electronic device 106b, the third wearable electronic device 106c, and the fourth wearable electronic device 106d. Information is transmitted to and received from the occupant devices only upon receiving consent from the users of the devices. As an example, the control system 108 may send information, commands, applications, media content and/or other data to the occupant devices, such as the first wearable electronic device 106a, the second wearable electronic device 106b, the third wearable electronic device 106c, and the fourth wearable electronic device 106d. As an example, the control system 108 may receive information from the occupant devices, such as signals that identify the location of each of the devices within the transportation device 100, and signals that describe the physiological states of the users of the devices.

FIG. 2 is a block diagram showing a communication system 220 for providing information from the control system 108 of the transportation device 100 to an emergency service provider system 222. The control system 108 of the transportation device 100 is configured to communicate with the emergency service provider system 222 using a long range wireless communications system, such as a cellular data communications system and/or a satellite data communications system. Such systems may be configured to allow transmission of data, including audio and video communications, between the control system 108 of the transportation device 100 and the emergency service provider system 222. Thus, the control system 108 of transportation device 100 may transmit information to and receive information from the emergency service provider system 222.

The emergency service provider system 222 is a computer implemented system that is associated with (e.g., used by) emergency service provider personnel in order to coordinate a response to the event. The emergency service provider system 222 includes conventional input and output devices to allow information to be presented to emergency service provider personnel. As one example, the emergency service provider system 222 may be used by a dispatch center to coordinate the response. As another example, the emergency service provider system 222 may be a mobile device used by personnel at the location of the event.

A wearable electronic device 206 represents one or more wearable electronic devices that are worn by one or more occupants of the transportation device 100, and is representative of the first wearable electronic device 106a, the second wearable electronic device 106b, the third wearable electronic device 106c, and the fourth wearable electronic device 106d. The description of the wearable electronic device 206 is therefore applicable to the first wearable electronic device 106a, the second wearable electronic device 106b, the third wearable electronic device 106c, and the fourth wearable electronic device 106d. The control system 108 of the transportation device 100 and the wearable electronic device 206 are configured to communicate withe each other. As an example, the control system 108 of the transportation device 100 and the wearable electronic device 206 may communicate with each other using a direct, short range wireless communication system that operates according to a suitable short range communications protocol. Alternatively, the control system 108 of the transportation device 100 and the wearable electronic device 206 may communicate indirectly (e.g., via a server that relays messages) using long range wireless communications.

In some implementations and/or situations, information transmitted from the wearable electronic device 206 to the control system 108 of the transportation device 100 is relayed to the emergency service provider system 222 by the control system 108 of the transportation device 100. In some implementations and/or situations, the wearable electronic device 206 communicates directly with the emergency service provider system 222 and transmits information to the emergency service provider system 222. In addition, in some implementations and/or situations, the wearable electronic device 206 receives information that is transmitted to it by the control system 108 of the transportation device 100 and relays that information to the emergency service provider system 222. Information may likewise be transmitted to the wearable electronic device 206 and optionally relayed to the control system 108 of the transportation device 100 by the emergency service provider system 222. As an example, the wearable electronic device 206 may be used to relay information between the control system 108 of the transportation device 100 when a long-range communications capability of the transportation device 100 is not functioning (e.g., damaged), and when the wearable electronic device 206 includes long-range communications functionality.

During normal operation of the transportation device 100, and independent of occurrence of an event during which the transportation device 100 has contacted an external object, the control system 108 may detect the presence of the wearable electronic device 206 (e.g., and additional wearable electronic devices) in the transportation device 100. The position of the wearable electronic device 206 relative to the transportation device 100 may also be determined by the control system 108 of the transportation device 100. As an example, the control system 108 of the transportation device 100 may be configured to determine the location of the wearable electronic device 206 within the passenger cabin of the transportation device 100, such as by identifying a seat (e.g., one of the first seat 104a, the second seat 104b, the third seat 104c, or the fourth seat 104d) that the occupant who is wearing the wearable electronic device 206 is seated in.

Detecting the presence of wearable electronic devices allows the transportation device 100 to determine the number of occupants that are located in the transportation device 100 and, optionally and only upon consent from the occupants, to determine their identities. Determining the position of wearable electronic devices relative to the transportation device 100 may allow information to be provided to the emergency service provider system 222 in a manner that allows identification of specific persons based on their locations in the transportation device 100.

The presence and/or positions of the wearable electronic devices, such as the wearable electronic device 206, may be determined based on signals received by the control system 108 from the wearable electronic devices. Specific examples of detection of the presence and the position of the wearable electronic device 206 by the control system 108 of the transportation device 100 will be described further herein.

The control system 108 of the transportation device 100 is configured to initiate communications with the emergency service provider system 222 in order to request assistance upon occurrence of an event during which the transportation device 100 has contacted (e.g., unintentionally and not as part of normal operations) an external object. The event may, for example, subject the transportation device 100 and its occupants to abnormal forces as a result of sudden deceleration or acceleration. The event may render some functions or systems of the transportation device 100 inoperable.

To allow the control system 108 of the transportation device 100 to initiate communications with the emergency service provider system 222, the control system 108 of the transportation device 100 is configured to detect the event in which the transportation device 100 has contacted an external object. As one example, the sensors 110 of the transportation device 100 may include contact-based sensors that are configured to directly sense contact of a portion (e.g., a body portion) of the transportation device 100 with the external object. Such sensors may, as examples, sense a change in air pressure in a chamber (e.g., a flexible chamber) as a result of the contact, sense deformation of a structure (e.g., using a bend sensor), sense a change in acceleration (e.g., using one or more accelerometers), or using another contact sensing technique. As another example, the sensors 110 of the transportation device may indirectly sense contact of the external object by interpretation of images, three-dimensional sensors outputs, or other sensors outputs that allow the control system 108 to determine the position and motion of the external object. Other sensing techniques may be used to detect contact of the external object with the transportation device 100.

In some implementations, the wearable electronic device 206 is configured to detect contact of the transportation device 100 with the external object. As an example, contact with an external object can be inferred based on acceleration of the wearable electronic device 206, such as by determining that acceleration of the wearable electronic device 206 has exceeded a threshold value that is beyond a magnitude of acceleration expected during normal movements of the occupant and the transportation device 100. In such implementations, the wearable electronic device 206 may be configured to initiate communications with the emergency service provider system 222, either directly, or through the control system 108 of the transportation device 100.

In response to the event, the transportation device 100 may use the sensors 110 to obtain information about the occupants of the transportation device 100. As an example, the sensors 110 may include one or more cameras that are configured to obtain images of the interior of the transportation device 100. The control system 108 of the transportation device 100 may obtain images of the interior of the transportation device 100, and identify a number of persons that are located in the transportation device 100, and their locations within the interior of the transportation device 100. As one example, images obtained by the control system 108 using the sensors 110 may be analyzed using a machine-vision system and or a machine learning based system to identify the presence of the occupants of within the transportation device 100, the number of the occupants, the locations of the occupants within the transportation device 100 (e.g., an identification of which seat they are sitting in), and an extent to which the occupants of the transportation device 100 are moving with respect to the transportation device 100. This information can be utilized by the control system 108 in assessing the conditions of the occupants, transmitted to the emergency service provider system 222, and/or utilized by the emergency service provider system 222 in assessing the conditions of the occupants.

In response to the event, the wearable electronic device 206 (as well as other wearable electronic devices worn by occupants of the transportation device 100) is configured to obtain information about the user of the wearable electronic device 206. To allow the wearable electronic device 206 to obtain the information about the user, the wearable electronic device includes sensors 224 that are configured to obtain information (e.g., in the form of sensor outputs) representing observations of states of the wearable electronic device 206, the user of the wearable electronic device 206, and/or the environment around the wearable electronic device 206.

The sensors 224 of may include an acceleration sensor 225a (e.g., one or more accelerometers) that is configured to measure acceleration of the wearable electronic device 206. As an example, the acceleration sensor 225a may be configured to measure acceleration of the wearable electronic device 206 in three linear degrees of freedom, and may further be configured to measure acceleration of the wearable electronic device 206 in three rotational degrees of freedom. Based on the acceleration measured by the acceleration sensor 225a, the forces experienced by the user of the wearable electronic device 206 may be estimated. In one implementation, the acceleration sensor 225a is part of an inertial measurement unit that includes one or more accelerometers, one or more gyroscopes, and/or one or more magnetometers to allow more complex and accurate characterization of the motion of the wearable electronic device 206. The sensors 224 may also include a satellite navigation system receiver 225b. The satellite navigation system receiver 225b is configured to receive signals from a satellite positioning system and to determine the location (e.g., expressed in geospatial coordinates) of the wearable electronic device 206 based on the received signals.

The sensors 224 may also include one or more physiological sensors that are configured to measure physiological states of the user of the wearable electronic device 206 and output physiological sensor information that describes one or more physiological states of the user. The physiological sensors may include a heart rate sensor 225c that is configured to monitor the heart rate of the user of the wearable electronic device 206. As one example, the heart rate sensor 225c may be implemented as an electrical monitor that uses one or more electrodes that are in contact with the user's body to detect heart electrical activity. As another example, the heart rate sensor 225c may be implemented using optical sensing technologies that include emission of visible and/or infrared light toward the user's body, and sensing and interpretation patterns of reflected visible and/or infrared light to measure heart rate. The physiological sensors may include a blood oxygenation sensor 225d that is configured to measure oxygenation of the blood of the user (e.g., peripheral oxygen saturation). The blood oxygenation sensor 225d may be implemented using optical sensing technologies that include emission of visible and/or infrared light toward the user's body, and sensing and interpretation patterns of reflected visible and/or infrared light to blood oxygenation. As an example, the heart rate sensor 225c and the blood oxygenation sensor 225d may be implemented in the form of a reflectance pulse oximetry device. The physiological sensors may also include a body temperature sensor 225e that is configured to measure the body temperature of the user of the wearable electronic device 206. Other physiological sensors may be included in the sensors 224 of the wearable electronic device 206 in order to measure physiological state information for the user of the wearable electronic device 206.

As described herein, one aspect of the present technology is the gathering and use of data available from specific and legitimate sources to facilitate emergency services under certain emergency conditions. The present disclosure contemplates that in some instances, this gathered data may include personal information data that uniquely identifies or can be used to identify a specific person. Such personal information data can include name, demographic data, physiological conditions, location-based data, online identifiers, telephone numbers, email addresses, home addresses, data or records relating to a user's health or level of fitness (e.g., vital signs measurements, medication information, exercise information), date of birth, or other personal information.

The present disclosure recognizes that the use of such personal information data, in the present technology, can be used to the benefit of users. For example, the personal information data can be used to facilitate emergency assistance to vehicle occupants. Entities that are responsible for the collection, analysis, disclosure, transfer, storage, or other use of such personal information data should comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominent and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking appropriate steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations that may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, users may select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified, via a user interface, upon an emergency event that their physiological information would be shared with emergency responders, and be provided with an opportunity to forgo sharing.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed embodiments, the present disclosure also contemplates that the various embodiments can also be implemented with options to reduce the amount of personal information shared.

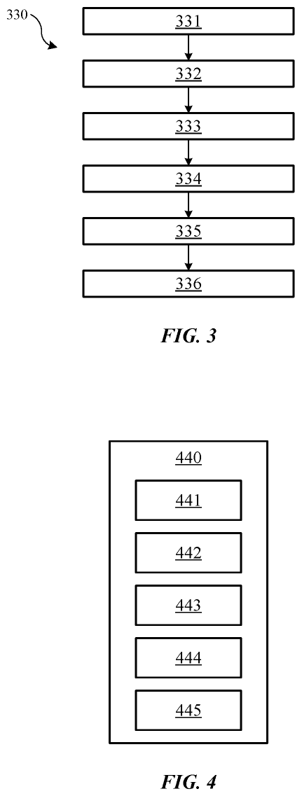

FIG. 3 is a block diagram of a process 330 for communicating with external services. The process 330 may be implemented in the context of the communication system 220, and the process 330 or portions of the process 330 may be implemented by one or more computing devices from the transportation device 100 (e.g., the control system 108 thereof), the wearable electronic device 206, and/or the emergency service provider system 222. The computing devices of these systems may be configured to perform the operations of the process 330, for example, using the example implementation of a computing device 440 of FIG. 4. As an example, the process 330 the steps thereof may be implemented in the form of computer program instructions that are executable by one or more computing devices, wherein the instructions, when executed by the one or more computing devices, cause the one or more computing devices to perform functions that correspond to the steps of the process. As an example, the process 330 and the steps thereof may be implemented in the form of a non-transitory computer-readable storage device including program instructions executable by one or more processors that, when executed, cause the one or more processors to perform operations that correspond to the steps of the process 330.

The process 330 allows a transportation device and/or wearable devices to obtain information about the occupants of the transportation device 100 and communicate that information to an emergency service provider system. In the illustrated implementation, the process 330 includes determining that a wearable is located in a transportation device and is travelling with the transportation device in operation 331, determining a position of a wearable electronic device relative to a transportation device in operation 332, detecting an event in which the transportation device has contacted an external object in operation 333, obtaining user state information from the wearable electronic device in operation 334, transmitting the position of the wearable electronic device relative to the transportation device and the user state information to an emergency service provider system in operation 335, and displaying the position of the wearable electronic device and user state information at the emergency service provider system 222 in operation 336. These operations will be described further herein using communication system 220 for context, including references to the transportation device 100, the wearable electronic device 206, and the emergency service provider system 222 as examples, but it should be understood that the operations of the process 330 may be implemented in other contexts including in the context of systems having different configurations.

Operation 331 includes determining that the wearable electronic device 206 is located in the transportation device 100 and is travelling with the transportation device 100. Presence of the wearable electronic device 206, or another electronic device associated with an occupant, may be determined based on signals received by the control system 108 of the transportation device 100 from the wearable electronic device 206 or other electronic device. In one implementation, determining that the wearable electronic device 206 is located inside the transportation device 100 and is travelling with the transportation device 100 is performed by comparison of the position of the wearable electronic device 206 relative to the transportation device 100 during a time period.

As one example, the wearable electronic device 206 may broadcast a signal indicating its presence, and that signal may be received by the control system 108 of the transportation device 100. If the signal indicating presence of the wearable electronic device 206 is received consistently over a significant period of time (e.g., received for longer than a predetermined time period), the control system 108 may determine that the wearable electronic device 206 is located in the transportation device 100 and is travelling with the transportation device 100. The presence of multiple electronic devices (e.g., including multiple wearable electronic devices worn by multiple occupants) within the transportation device 100 may be detected in the same manner.

As another example, the position of the transportation device 100 and the position of the wearable electronic device 206 may be determined independently, such as by use of satellite positioning system receivers associated with each of the transportation device 100 and the wearable electronic device 206. The positions may be shared between the devices or shared with another device (e.g., a server), thereby allowing the relative position of the transportation device 100 with respect to the wearable electronic device 206 to be seen over time. Thus, the determination as to whether the wearable electronic device 206 is located in and/or travelling with the transportation device 100 may be made by comparison of the position of the wearable electronic device 206 relative to the transportation device 100 during a time period. As an example, if a distance between the position of the wearable electronic device 206 and the position of the transportation device is less than a threshold distance for at least a predetermined time period (e.g., the distance between the two remains less than the threshold value during the predetermined time period), it can be inferred that the wearable electronic device 206 is located inside the transportation device 100 and is travelling with the transportation device 100. As another example, if the difference in position between the position of the wearable electronic device 206 and the position of the transportation device is less than a threshold value for at least a predetermined time period (e.g., the distance between the two changes by less than the threshold value during the predetermined time period), it can be inferred that the wearable electronic device 206 is located inside the transportation device 100 and is travelling with the transportation device 100. As another example, a motion pattern (e.g., including multiple measurements representing acceleration at different points in time) of the transportation device 100 and a motion pattern of the wearable electronic device 206 during a time period may be compared to determine whether two motion patterns are consistent with each other (e.g., agree within at least a threshold amount), and the wearable electronic device 206 is determined to be located in and travelling with the transportation device 100 if the two motion patterns are consistent.

As another example, the wearable electronic device 206 may be registered with the transportation device 100 to indicate the presence of the wearable electronic device 206 and its user within the transportation device 100. The wearable electronic device 206 may be registered with the transportation device 100 by pairing or a similar process. As an example, wearable electronic device 206 may detect proximity to the transportation device 100, and prompt the user to register the wearable electronic device 206 with the control system 108 of the transportation device 100, with consent provided by the user of the wearable electronic device. During normal operation of the transportation device 100, registration of the wearable electronic device 206 with the transportation device 100 may allow the wearable electronic device 206 to output information about operation of the transportation device 100, allow the user to control operation of various systems and features of the transportation device, or implement other functionality that includes communications between the transportation device 100 and the wearable electronic device 206.

Operation 332 includes determining the position of the wearable electronic device 206 relative to the transportation device 100. Operation 332 may be performed by the control system 108 of the transportation device 100, for example, using signals received from the wearable electronic device 206 and/or using information obtained from the sensors 110 of the transportation device 100.

In operation 332, the position of the wearable electronic device 206 (and/or other wearable electronic devices) relative to the transportation device 100 may be determined. As one example, the position of the wearable electronic device 206 relative to the transportation device 100 may be determined based on one or more signals received by the control system 108 from the wearable electronic device 206. As another example, the position of the wearable electronic device 206 relative to the transportation device 100 may be determined based on comparison of satellite positioning system coordinates for the wearable electronic device 206 and the transportation device 100. As another example, the position of the wearable electronic device 206 relative to the transportation device 100 may be determined based on outputs from the sensors 110 (e.g., images from cameras) of the transportation device 100, where the outputs from the sensors 110 represent observations of the passenger cabin of the transportation device 100.

As an example, determining the position of the wearable electronic device 206 relative to the transportation device 100 in operation 332 may include receiving, by the control system 108 of the transportation device 100, a location signal from the wearable electronic device 206. The location signal received from the wearable electronic device 206 may be a signal that provides a sufficient basis from which to determine a distance and angle between a transmitter and receiver pair, such as a transmitter (e.g., one or more transmitters) included in the wearable electronic device 206 and a receiver (e.g., one or more receivers) included in the transportation device 100. As an example, the transportation device 100 and the wearable electronic device 206 may include radio frequency location sensing components that allow the distance and angle between a transmitter and receiver to be determined (e.g., by triangulation), thereby allowing the control system 108 to determine the location of the wearable electronic device 206 in the transportation device 100.

As another example, a position of the transportation device 100 and a position of the wearable electronic device 206 may be determined independently, such as by use of satellite positioning system receivers associated with each of the transportation device 100 and the wearable electronic device 206. A difference between the two positions (e.g., the average position difference during a time period) may be utilized as a basis for estimating the position of the wearable electronic device 206 relative to the transportation device. As an example, the two positions may be expressed in terms of geospatial coordinates.

In operation 332, determining the position of the wearable electronic device 206 may include determining a location of the wearable electronic device 206 within the passenger cabin of the transportation device 100. As an example, the of the wearable electronic device 206 may be determined with sufficient accuracy to identify the seat that the user (e.g., one of the first occupant 102a, the second occupant 102b, the third occupant 102c, or the fourth occupant 102d) wearing the wearable electronic device 206 is sitting in. As previously described, an angle and distance between a transmitter or receiver of the transportation device relative to the wearable electronic device 206 can be determined based on triangulation of signals or another method, and this angle and distance can be used to match a measured location of the wearable electronic device 206 to one of the seats (e.g., the first seat 104a, the second seat 104b, the third seat 104c, or the fourth seat 104d) of the transportation device 100 by proximity (e.g., shortest distance between the wearable electronic device 206 and one of the seats and/or a distance between the wearable electronic device 206 and one of the seats being lower than a threshold distance).

In addition to use of location signals to determine the position of the wearable electronic device 206 within the transportation device 100, images obtained by a camera from the sensors 110 of the transportation device 100 may also be used to determine position. As an example, images taken inside the passenger cabin of the transportation device 100 by a camera from the sensors 110 can be analyzed to identify persons that are present in the passenger cabin and their respective locations. This information can be used to identify plausible locations for the wearable electronic device 206, to match the wearable electronic device 206 to one of the occupants, and/or to determine the identity of an occupant who is wearing the wearable electronic device 206.

Operation 333 includes detecting an event in which the transportation device 100 has contacted an external object. Operation 333 may be performed by the control system 108 of the transportation device 100, for example, using information from the sensors 110 of the transportation device 100. As previously described, the event may be detected directly using contact-based sensors, or may be detected indirectly by detecting and tracking motion of the external object relative to the transportation device using images, three-dimensional sensors outputs, or other sensors outputs. Other sensing techniques may be used.

Operation 334 is performed in response to detection of the event in operation 333, and includes obtaining state information about a user of the wearable electronic device 206. In operation 334, the user of the wearable electronic device 206 is assumed to be one of the occupants of the transportation device 100, based on the determination that the wearable electronic device 206 is located in and travelling with the transportation device 100 in operation 331.

Obtaining state information about the user of the wearable electronic device 206 in operation 334 may include obtaining motion information describing motion of the user of the wearable electronic device 206 during the event. As an example, the motion information describing motion of the user of the wearable electronic device 206 during the event that is obtained in operation 334 may include an acceleration value measured by the acceleration sensor 225a of the wearable electronic device 206 during the event. The motion information may indicate, for example, a maximum force experienced by the user of the wearable electronic device 206 during the event. The motion information may include, for example, a series of measurements that represent forces experienced by the user of the wearable electronic device 206 during the event. Alternatively, the motion information may include information that describes the motion of the user of the wearable electronic device 206 during the event, expressed in any suitable form.

Obtaining state information about the user of the wearable electronic device 206 in operation 334 may include obtaining physiological sensor information from one or more physiological sensors of the wearable electronic device 206. The physiological sensor information describes one or more physiological states of the user of the wearable electronic device 206. The physiological sensor information obtained in operation 334 may be useful, for example, in determining, whether any current physiological states of the user deviate from typical physiological states (e.g., whether a current measured value falls inside of an expected range or outside of an expected range). As an example, the physiological sensor information that is obtained by the wearable electronic device 206 may include heart rate information for the user of the wearable electronic device 206 that is obtained by the heart rate sensor 225c of the wearable electronic device 206. As an example, the physiological sensor information that is obtained by the wearable electronic device 206 may include oxygen level information for the user of the wearable electronic device 206 that is obtained by the blood oxygenation sensor 225d of the wearable electronic device 206. As an example, the physiological sensor information that is obtained by the wearable electronic device 206 may include body temperature information for the user of the wearable electronic device 206 that is obtained by the body temperature sensor 225e of the wearable electronic device 206.

Obtaining state information about the user of the wearable electronic device 206 in operation 334 may include obtaining cognitive function information by prompting the user of the wearable electronic device 206 to complete one or more tasks using the wearable electronic device 206. Prompting the user to complete tasks that evaluate cognitive function may include displaying text and/or images on a display screen of the wearable electronic device 206, and may include receiving inputs using one or more input devices of the wearable electronic device 206. The inputs may include a touch input received by a touch-sensitive input device of the wearable electronic device 206. The inputs may include a physical button press input received by a physical button of the wearable electronic device 206. The inputs may include a physical knob rotation received by a physical knob of the wearable electronic device 206. The inputs may include a verbal input received by an audio input device (e.g., a microphone) of the wearable electronic device 206.

As one example, the wearable electronic device 206 may prompt the user to complete a task that includes answering simple questions (either displayed as text or output as audio). The answers to the question can be obtained by the sensors 224 of the wearable electronic device 206. The substantive correctness of the answers to the questions may be determined to understand whether the user is responsive and alert. The question may be answered by speaking, which allows the wearable electronic device 206 to capture a speech audio sample that can be analyzed to determine whether (e.g., by audio analysis methods or using a trained machine learning model) the user's speech indicates any decrease in cognitive function. Examples of simple questions that the user may be prompted to answer include saying their name or answering a simple math problem. As another example, the wearable electronic device 206 may prompt the user to complete a task that includes operating an input device of the wearable electronic device 206, such as pressing a button when an image appears or rotating a knob to cause rotation of an image (e.g., an icon) to a predetermined orientation. As another example, the wearable electronic device 206 may prompt the user to complete a task that includes operating an input device of the wearable electronic device 206 when a sound is played by an audio output device of the wearable electronic device 206.

The wearable electronic device 206 is configured to output cognitive function information based on the assessment in which the user completes tasks that evaluate cognitive function. In one implementation, the cognitive function information may indicate whether the person is responsive. In one implementation, the cognitive function information may include information indicating the tasks performed by the user and whether they were performed appropriately. In one implementation, the cognitive function information may include a cognitive function score that is determined, for example, by awarding points for successful completion of tasks, and determining the cognitive function score based on the number of points awarded (e.g., by outputting a percentage of points awarded relative to total points available).

Obtaining state information about the user of the wearable electronic device 206 in operation 334 may additionally be performed using the sensors 110 of the transportation device 100. As an example, images of the passenger cabin of the transportation device 100 may be obtained using cameras that are included in the sensors 110 of the transportation device 100. These images may be interpreted (e.g., using a machine vision system and/or a trained machine learning model) to identify user of the wearable electronic device 206 and/or other occupants of the transportation device 100 in order to determine information about their condition (e.g., movement, responsiveness, etc.). Information resulting from this analysis and/or the images may be transmitted to the emergency service provider system 222. As another example, microphones included in the sensors 110 of the transportation device 100 may be used to obtain audio that can be sent to the emergency service provider system 222 and/or used to establish audio communications with the occupants of the transportation device.

Operation 335 includes transmitting information to the emergency service provider system 222. Operation 335 may be performed by the control system 108 of the transportation device 100. The information that is transmitted in operation 335 may include the position of the wearable electronic device 206 relative to the transportation device 100. The information that is transmitted in operation 335 may include the identity of the user of the wearable electronic device 206. The information may include user state information about the user of the wearable electronic device 206. The user state information may include the motion information, the physiological sensor information, and the cognitive function information obtained in operation 335. Other information about the user of the wearable electronic device 206 may also be transmitted to the emergency service provider system 222 in operation 335.

In some implementations, the transmission of information to the emergency service provider system 222 that is performed in operation 335 may instead be performed by the wearable electronic device 206. As an example, the control system 108 of the transportation device 100 may determine that it is unable to contact the emergency service provider system 222 directly, and may request that the wearable electronic device 206 transmit the information, which may be provided to the wearable electronic device 206 for transmission by the control system 108 and/or by other wearable electronic devices that are located in the transportation device 100.

In some implementations, transmitting the user state information to the emergency service provider system 222 in operation 335 is performed in response to determining non-compliance of the cognitive function information relative to a standard. If the standard is met, this indicates that the evaluation of 334 found some evidence of decreased cognitive function, and transmission of information to the emergency provider system 222 according to operation 335 may be performed automatically in response. If the standard is met, this indicates that the evaluation of 334 found no significant evidence of decreased cognitive function, in which case, transmission of information to the emergency provider system 222 is not performed automatically or is only performed in response to a further input from the user expressing an intention to do so. As an example, the tasks used to evaluate cognitive function in operation 334 may each include a standard that is used to evaluate the user's performance of the task as compliant, indicating no significant evidence of decreased cognitive function, or non-compliant, indicating some evidence of decreased cognitive function. A standard may for determining compliance may include comparing a time required for performance of a task by the user to a predetermined value. A standard may include determining whether a question was answered correctly. A standard may include scoring multiple evaluations (e.g., by awarding a certain number of points for compliance as to each evaluation) and totaling the score in order to compare the score to a predetermined threshold value that corresponds to compliance with the standard. Other methods may be used. Thus, compliance or non-compliance of the result of the cognitive function evaluation with the standard may be used to determine whether operation 335 is performed automatically.

Operation 336 includes displaying information using the emergency service provider system 222. The information displayed includes some or all of the information obtained in operation 334 and transmitted in operation 335, as previously described. The information displayed using the emergency service provider system 222 in operation 336 may include the position of the wearable electronic device 206 relative to the transportation device 100, and may include the positions of other devices. The information displayed using the emergency service provider system 222 in operation 336 may include the user state information for the user of the wearable electronic device 206, and may include user state information for users of other devices (e.g., wearable electronic devices worn by other occupants of the transportation device 100). If available, the identities of users of the devices may be displayed in association with the position information and the user state information, consistent with the user's preferences for sharing such information.

It should be understood that the process 330 may be performed for multiple occupants through use of their respective wearable electronic devices. Thus, for example, operations 331, 332, and 334 may be performed with respect to two or more wearable electronic devices that are located in the transportation device 100 and which are each associated with a different occupant of the transportation device 100. Thus, information regarding other occupants of the transportation device 100, including information obtained from their respective wearable electronic devices, may also be transmitted in operation 335 and displayed in operation 336 in the same manner described with respect to the wearable electronic device 206.

Accordingly, an implementation of the process 330 may include determining, by the control system 108 of a transportation device 100, the positions of wearable electronic devices relative to the transportation device 100, and detecting, by the control system 108 of the transportation device 100, an event in which the transportation device 100 has contacted an external object. In response to the event, the implementation of the process 330 may include obtaining user state information about users of the wearable electronic devices, wherein obtaining the user state information includes obtaining motion information describing motion of each of the users during the event from a respective wearable electronic device, obtaining physiological sensor information describing one or more physiological states of each of the user from the respective wearable electronic device, and obtaining cognitive function information by prompting each user to complete one or more tasks using the respective wearable electronic device.

It should be noted that while the description above is made with respect to wearable electronic devices, the functions described with respect to the process 330 may be implemented using a non-wearable electronic device that is carried by an occupant of the transportation device 100, such as a smart phone. In such an implementation, obtaining some information, such as physiological state information, may be limited according to the sensing capabilities of the particular device, but motion information and state information for the user may regardless be obtained and transmitted.

The process 330 may also include, subsequent to detection of the event in operation 333, identifying other devices that are located near the transportation device 100, and which may be associated with other persons involved in the event. Information may be obtained, transmitted, and displayed using the emergency service provider system 222 in the same manner as described with respect to the devices located in the transportation device 100, consistent with user privacy configurations.

In some implementations of the process 330, some features, components, and/or operations may be omitted.

FIG. 4 is a block diagram of an example of a computing device 440 that can be used to implement one or more computing devices that execute the systems and processes described herein, such as computing devices incorporated in the control system 108 of the transportation device 100, the wearable electronic device 206, the emergency service provider system, and/or other systems. The computing device 440 may include a processor 441, a memory 442, a storage device 443, one or more input devices 444, and one or more output devices 445. The computing device 440 may include a bus or a similar device to interconnect the components for communication. The processor 441 is operable to execute computer program instructions and perform operations described by the computer program instructions. As an example, the processor 441 may be a conventional device such as a central processing unit. The memory 442 may be a volatile, high-speed, short-term information storage device such as a random-access memory module. The storage device 443 may be a non-volatile information storage device such as a hard drive or a solid-state drive. The input devices 444 may include any type of human-machine interface such as buttons, switches, a keyboard, a mouse, a touchscreen input device, a gestural input device, or an audio input device. The output devices 445 may include any type of device operable to provide an indication to a user regarding an operating state, such as a display screen or an audio output.

What is claimed is:

1. A method, comprising:
detecting, by a control system of a transportation device, an event in which the transportation device has contacted an external object;
in response to the event, obtaining state information about a wearer of a wearable electronic device, wherein obtaining the state information includes:
obtaining motion information describing motion of the wearer of the wearable electronic device during the event, and
obtaining physiological sensor information from one or more physiological sensors of the wearable electronic device, the physiological sensor information describing one or more physiological states of the wearer of the wearable electronic device; and
transmitting, by the control system of the transportation device, the user state information about the wearer of the wearable electronic device to an emergency service provider system, the user state information including the motion information, and the physiological sensor information.

2. The method of claim 1, further comprising:
determining, by a control system of a transportation device, a position of a wearable electronic device relative to the transportation device; and
transmitting, by the control system of the transportation device, the position of the wearable electronic device relative to the transportation device to the emergency service provider system,
wherein determining the position of the wearable electronic device relative to the transportation device includes receiving, by the control system of the transportation device, a location signal from the wearable electronic device.

3. The method of claim 1, further comprising:
determining that the wearable electronic device is present in the transportation device based on a wireless communication received by the transportation device from the wearable electronic device.

4. The method of claim 1, wherein the motion information describing motion of the user of the wearable electronic device during the event includes an acceleration value measured by an acceleration sensor of the wearable electronic device during the event.

5. The method of claim 1, wherein the physiological sensor information includes at least one of heart rate information for the user of the wearable device or oxygen level information for the user of the wearable electronic device.

6. The method of claim 1, wherein:
obtaining the state information further includes obtaining cognitive function information by prompting the wearer of the wearable electronic device to complete one or more tasks using the wearable electronic device, and
transmitting the user state information to the emergency service provider system comprises transmitting the cognitive function information to the emergency provider system.

7. The method of claim 1, wherein:
obtaining the state information further includes obtaining cognitive function information by prompting the wearer of the wearable electronic device to complete one or more tasks using the wearable electronic device, and
transmitting the user state information to the emergency service provider system is performed in response to determining non-compliance of the cognitive function information relative to a standard.

8. A non-transitory computer-readable storage device including program instructions executable by one or more processors that, when executed, cause the one or more processors to perform operations, the operations comprising:
detecting, by a control system of a transportation device, an event in which the transportation device has contacted an external object;
in response to the event, obtaining state information about a wearer of a wearable electronic device, wherein obtaining the state information includes:
obtaining motion information describing motion of the wearer of the wearable electronic device during the event, and
obtaining physiological sensor information from one or more physiological sensors of the wearable electronic device, the physiological sensor information describing one or more physiological states of the wearer of the wearable electronic device; and
transmitting, by the control system of the transportation device, the user state information about the wearer of the wearable electronic device to an emergency service provider system, the user state information including the motion information, and the physiological sensor information.

9. The non-transitory computer-readable storage device of claim 8, the operations further comprising:
determining, by a control system of a transportation device, a position of a wearable electronic device relative to the transportation device; and
transmitting, by the control system of the transportation device, the position of the wearable electronic device relative to the transportation device to the emergency service provider system,
wherein determining the position of the wearable electronic device relative to the transportation device includes receiving, by the control system of the transportation device, a location signal from the wearable electronic device.

10. The non-transitory computer-readable storage device of claim 8, the operation further comprising:
determining that the wearable electronic device is present in the transportation device based on a wireless communication received by the transportation device from the wearable electronic device.

11. The non-transitory computer-readable storage device of claim 8, wherein the motion information describing motion of the user of the wearable electronic device during the event includes an acceleration value measured by an acceleration sensor of the wearable electronic device during the event.

12. The non-transitory computer-readable storage device of claim 8, wherein the physiological sensor information includes at least one of heart rate information for the user of the wearable device or oxygen level information for the user of the wearable electronic device.

13. The non-transitory computer-readable storage device of claim 8, wherein:
obtaining the state information further includes obtaining cognitive function information by prompting the wearer of the wearable electronic device to complete one or more tasks using the wearable electronic device, and
transmitting the user state information to the emergency service provider system comprises transmitting the cognitive function information to the emergency provider system.

14. The non-transitory computer-readable storage device of claim 8, wherein:
obtaining the state information further includes obtaining cognitive function information by prompting the wearer of the wearable electronic device to complete one or more tasks using the wearable electronic device, and
transmitting the user state information to the emergency service provider system is performed in response to determining non-compliance of the cognitive function information relative to a standard.

15. An apparatus, comprising:
a memory; and
one or more processors that are configured to execute instructions that are stored in the memory, wherein the instructions, when executed, cause the one or more processors to:
detect, by a control system of a transportation device, an event in which the transportation device has contacted an external object;
in response to the event, obtain state information about a wearer of a wearable electronic device, wherein the instructions to obtain the state information include instructions that cause the one or more processors to:
obtain motion information describing motion of the wearer of the wearable electronic device during the event, and
obtain physiological sensor information from one or more physiological sensors of the wearable electronic device, the physiological sensor information describing one or more physiological states of the wearer of the wearable electronic device; and
transmit, by the control system of the transportation device, the user state information about the wearer of the wearable electronic device to an emergency service provider system, the user state information including the motion information, and the physiological sensor information.

16. The apparatus of claim 15, wherein the instructions further cause the one or more processors to:
determine, by a control system of a transportation device, a position of a wearable electronic device relative to the transportation device; and
transmit, by the control system of the transportation device, the position of the wearable electronic device relative to the transportation device to the emergency service provider system,
wherein determining the position of the wearable electronic device relative to the transportation device includes receiving, by the control system of the transportation device, a location signal from the wearable electronic device.

17. The apparatus of claim 15, wherein the instructions further cause the one or more processors to:
determine that the wearable electronic device is present in the transportation device based on a wireless communication received by the transportation device from the wearable electronic device.

18. The apparatus of claim 15, wherein the motion information describing motion of the user of the wearable electronic device during the event includes an acceleration value measured by an acceleration sensor of the wearable electronic device during the event.

19. The apparatus of claim 15, wherein the physiological sensor information includes at least one of heart rate information for the user of the wearable device or oxygen level information for the user of the wearable electronic device.

20. The apparatus of claim 15, wherein the instructions further cause the one or more processors to:
obtain the state information further includes obtaining cognitive function information by prompting the wearer of the wearable electronic device to complete one or more tasks using the wearable electronic device, and
transmit the user state information to the emergency service provider system comprises transmitting the cognitive function information to the emergency provider system.
